(19) United States
(12) Patent Application Publication
(10) Pub. No.: US 2018/0310979 A1
(43) Pub. Date: Nov. 1, 2018

(54) METHOD AND APPARATUS FOR USE OF ICE CRYSTALS IN AESTHETIC AND COSMETIC PROCEDURES

(71) Applicants: Omer Peled, Haifa (IL); A. Jason Mirabito, Derry, NH (US)

(72) Inventors: Omer Peled, Haifa (IL); A. Jason Mirabito, Derry, NH (US)

(21) Appl. No.: 15/977,328

(22) Filed: May 11, 2018

Related U.S. Application Data
(63) Continuation of application No. PCT/US2016/061417, filed on Nov. 10, 2016.
(60) Provisional application No. 62/254,862, filed on Nov. 13, 2015.

(51) Int. Cl. A61B 18/02; A61B 17/54

(52) U.S. Cl. CPC A61B 18/0218; A61M 35/00; A61B 17/545

(57) ABSTRACT

A device combines skin tissue cooling and microdermabrasion and includes: a source of ice crystals; a mechanism for propelling the ice crystals at a plurality of velocities; a controller, the controller being programmable to increase or decrease the velocity of the propelled ice crystals; the controller controls the velocity of the ice crystals to range from low speed to impinge upon and cool the skin tissue to a higher velocity to cause microdermabrasion of the skin tissue.

### RELATED APPLICATIONS

This application is a continuation of PCT Application No. PCT/US2016/061417, filed Nov. 10, 2016, which is related to and claims priority from U.S. Provisional Application Ser. No. 62/254,862, filed Nov. 13, 2015, the entirety of which is incorporated by reference herein.

### BACKGROUND OF THE PRESENT INVENTION

This invention relates to the use of ice crystals in various forms in the providing of aesthetic and cosmetic procedures. The use of ice crystals for industrial purposes is well-known particularly for finishing and deburring products. At least one patent, U.S. Pat. No. 8,430,722 briefly addresses the possibility of using ice crystals for medical procedures but few details of the apparatus and method of application are provided. The aforementioned patent discloses the use of ice crystals, usually in the form of dry ice, for the purpose of treatment of diseases of the skin, including cosmetic treatment. The ice crystals are propelled by a suitable means to impinge on the skin within a certain parameters set by the delivery device.

It is also known in the aesthetic and cosmetic fields of treatment to treat skin conditions, such as wrinkles or discolorations by way of example only, with one or more of light (in the form of laser light, intense pulsed light (IPL) and LED light, as well as radio frequency (RF) energy and ultrasound (US) energy. A very large industry has been built around these technologies. One form of light treatment is the so-called fractional treatment, in which a plurality of wounds, some ablative, others non-ablative are made into the skin surface but spaced apart from one another. It has been found that such treatments are particularly efficacious in causing collagen regrowth and thus reductions of wrinkles or other skin conditions.

Heretofore, the inventors are not aware of a combination of such ice crystal treatments and light/RF/US treatments being combined. It is to this aspect that the present invention is in part directed in combination with the disclosure of devices and modalities of delivery of ice crystals alone or in combination with light/RF/US. The following are some examples of the inventive concepts. Ice crystals or pellets as described herein may be of so-called dry ice which is a solid form of CO2 in the form of pellets or blocks of dry ice, dry ice “snow” which is dry ice generated from compressed CO2, may be H₂O crystals, or may be either of those combined with another substance such as a drug, medicine, cosmetics, skin nutrition elements such as vitamins and minerals, nano particles such as Silver, Gold, Titanium dioxide, Zinc oxide, Quantum dots or Carbon based nanoparticles, anesthetic substance to give just a few examples. Other materials may be crystallized and utilized with the present invention.

Energy based treatments such as, for example, skin rejuvenation, hair removal or fat reduction may be achieved through a selective treatment, in which a specific interaction between the energy and a target tissue or chromospheres is achieved. A fundamental element in these treatments is heating a target tissue. Alternatively, a bulk treatment to a tissue region without any selectivity may also be applied to achieve some clinical results. Another strategy to achieve local effect in a target tissue could be localized energy application such as a focused light beam, a laser beam, RF, microwaves or focused ultrasound which may provide non-invasively modalities to treat target tissue in the skin or below. A minimal invasive approach to localize energy in a target tissue could be achieved with ablative energy sources which intend to penetrate the skin and deliver energy into the target tissue. Ablative fractional laser, ablative fractional RF or micro-needles are examples for minimal invasive solutions which may localize energy in target tissues at different depth in or below the skin.

A more invasive methodology to localize energy in target skin layers, for example, in known by using invasive cannula. Fat liposuction is an invasive procedure which practices a cannula having an energy source which is inserted into the fat layer underneath the skin in order to melt or rapture fat tissue. Alternatively, brachytherapy provides localized irradiation of target cancerous tissue with radioactive seeds. High dose treatment (HDT) to a tissue volume is provided by inserting multiple needles into the target tissue and by sliding the seeds along the needle portion inside the target tissue. In a low dose treatment (LDT) the seeds are implanted permanently in the tissue volume. In a brachytherapy device, a loader loads radioactive seeds into an array of flexible micro tubes. At the distal end of the micro tube there is a narrow solid needle. The array of needles is inserted through a two-dimensional rigid net of holes which help the physician to target specific region in the target tissue based on an image and pre-analysis done for the target tissue. According to a treatment plan radioactive seeds are distributed in a tissue volume so that radiation coverage is optimized both spatially and along the time domain.

Cooling the skin is a known strategy in many irradiating procedures which target inner layers of the skin. Cooling the upper surface of the skin is used in order to enhance selectivity and protect the upper layers of the skin which are not the target of the treatment. The natural thermal gradient across the skin is characterized in that it is hotter inside and colder on the surface. When high amount of energy is delivered topically, the upper layers of the skin may be exposed to a high energy fluence which may damage the skin. The high fluence may be required in order to deliver enough energy into the target tissue. Energy is lost along its propagation in the tissue due to many factors and mechanisms. Cooling the skin may allow delivering higher energy fluences across the skin while protecting its upper layers. This skin cooling may be combined with microdermabrasion in a single device that is able to be adjusted from a gentler, less rapid impingement on skin tissue to cool the skin to a state in which the impingement is less gentle and may be strong enough and with an ice crystal velocity and size sufficient to cause microdermabrasion of the skin tissue.

Cryosurgery is also known in the prior art. A review of Keloid scar treatment with a cryo-needle intralesionally is described for example in a paper “Use of Intralesional Cryosurgery as an Innovative Therapy for Keloid Scars and a Review of Current Treatments” by Goldenberg et al. Prof. Har Shai teaches translesion treatment of hypertrophic and keloid scars in a presentation available at the following link http://www.scar-club.com/pdf/powerpoint/Har%20Shai%20Yaron.pdf. In his analysis, Prof. Har Shai shows the advantage of the intralesion solution over a contact cooling probe.

Introducing a cold probe topically on the skin or any other tissue, in order to cool a target tissue below dictates a problematic temperature gradient across that tissue. Based on the nature of cooling, which is actually by the law of physics heat pumping, cooling an inner tissue to a certain temperature requires a temperature gradient across the tissue which is colder at the point of probe contact. Otherwise heat will not be pumped out from the target tissue. As mentioned above, in the case of heating a target tissue, it is possible to deliver more energy through a tissue to a target tissue while cooling the upper layers of the tissue to protect it from high energy fluence. However, when it comes to a treatment which aims to cool a target tissue, it is impossible to “bring” more cold into the tissue while heating the upper layer of tissue in order to protect it from over exposure to the cold. The minute the outer layer of the tissue is hotter than the temperature of the tissue below, the driving force for pumping the heat out of the target tissue is stopped and no further temperature reduction to a target tissue may be achieved. This is another aspect addressed by the present invention.

Galil Medical is a company which provides cryo-needles technology which is capable to cool only a distal tip of the needle, thus improving the localization of the cooling treatment. Cryogenic balloon catheters are used today by Medtronic and Boston Scientific for the treatment of atrial fibrillation. Moreover, cooling as a treatment modality in aesthetic medicine gains more attention recently. Zeltiq Inc. practices a non-invasive tissue bulk cooling technology for the indication of fat reduction as disclosed in U.S. Pat. No. 7,367,341.

It is another object of the current invention to provide new and alternative systems and the methods to controllably cool areas of skin or other tissue rejoins as will be described below.

### SUMMARY OF THE PRESENT INVENTION

In an aspect, a device which combines skin tissue cooling and microdermabrasion includes: a source of ice crystals; a mechanism for propelling the ice crystals at a plurality of velocities; a controller, the controller being programmable to increase or decrease the velocity of the propelled ice crystals; the controller controls the velocity of the ice crystals to range from low speed to impinge upon and cool the skin tissue to a higher velocity to cause microdermabrasion of the skin tissue.

In another aspect, a device for treatment of skin tissue includes: a source of ice crystals; a mechanism for: (a) propelling the ice crystals, (b) controlling the size and shape of the ice crystals, and (c) fractionating the ice crystals delivery; a controller; the ice crystals are propelled, under the control of the controller, to impinge on the skin tissue surface in a spaced-apart, fractionated pattern. In addition, the controller controls the mechanism for propelling the velocity of the ice crystals impinging on the skin tissue with sufficient velocity to penetrate the skin tissue surface.

In yet another aspect, a device for the treatment of skin tissue includes: a source or ice crystals; a mechanism for propelling the ice crystals; a controller; a source of electromagnetic energy in the vicinity of the mechanism for propelling the ice crystals; wherein the controller controls: (a) the mechanism for propelling the ice crystals and (b) the source of electromagnetic energy. The controller causes the mechanism to propel ice crystals at the skin tissue one of before, during or after the source of electromagnetic energy is activated by the controller to impinge electromagnetic energy towards the skin tissue. The source of electromagnetic energy is one or more of: laser energy, IPL, LED, Microwave, RF energy and ultrasonic energy and the source of electromagnetic energy may be fractionated energy.

In a further aspect, a device for the treatment of tissue includes: one or more hollow needles; a source of ice crystals, the ice crystals being sized to fit within the one or more hollow needles; a controller; a mechanism for propelling the ice crystals through the one or more hollow tubes. The controller causes the mechanism to propel the ice crystals through the one or more hollow needles, out the distal end of the one or more needles and into the tissue. In addition, the one or more ice crystals may be preloaded into the one or more hollow needles.

### BRIEF DESCRIPTION OF THE DRAWINGS

FIGS. 1 and 2 illustrate the structure and operation of two embodiments of the present invention.

FIG. 3 illustrates a combination of ice crystals and a source of laser light energy.

FIG. 4 illustrates a combination of ice crystals with a source of RF energy.

FIG. 5 illustrates a type of fractionated ice blasting device.

FIG. 6 illustrates another type of fractionated ice blasting device.

FIGS. 7A-7D illustrate the penetration of ice crystals into skin tissue.

FIGS. 8a and 8b illustrate the use of ice crystals in combination with hollow needles in an ice-seeding device and procedure.

FIG. 9 illustrates the use of ice crystals in the treatment of Keloid scars.

### DETAILED DESCRIPTION OF THE PRESENT INVENTION

### Removal of the Stratum Corneum (SC)

The stratum corneum is the upper layer of the skin that contains 15-20 layers of dead cells. The thickness of the stratum corneum varies between 10-40 μm depending on the person and in which place on the body it is located. The main function of the stratum corneum is to protect the deeper layers of the skin from injuries and bacterial invasion. However, the stratum corneum is likewise considered a strong barrier to the diffusion of any compounds and drugs through the lower skin layers.

The stratum corneum, as a dry layer of dead cells has a different characteristic than the living tissue underneath. In certain procedures, there is a need to remove the stratum corneum in order to improve the energy coupling of skin tissue with an energy source. For example, RF or Ultrasound may be coupled more efficiently to living tissue (which contains high percentage of water) than it can be coupled to the SC. Therefore, in order to overcome coupling problems with the SC, some devices operate by increasing the energy provided in order to penetrate or ablate the SC; some other devices attempt to improve the coupling with a coupling material such as an ultrasound gel. According to an embodiment of the present invention, an ice blasting device is disclosed which is configured to remove the SC from the skin in order to expose the living skin. The exposed living skin may be further treated by energy based treatments such as light, RF or US with lower energies should the SC were still in place.

Moreover, the effect of blasting the SC with ice particles will simultaneously reduce the temperature of the exposed tissue and some depth below the exposed tissue. Therefore, another aspect of the invention is removing the SC and simultaneously reducing temperature of the tissue. The energy of the ice particles according to some aspects of this invention, as will be described below, can be controlled so that different tissue effects may be achieved. A non-blasting cooling treatment profile may also be achieved and may allow the cooling of the skin for a period of time without causing any blasting or skin peeling effect. This mode of the operation may keep the skin at a certain temperature range as needed for a required period of time. This can be achieved by a using free-hand paint brush mode handpiece which allows a physician to keep a tissue region in the desired temperature range or through use of a scanner device which may automatically scan one more nozzles which are configured to blow a stream of ice particles.

A thermal imaging system may also be used according to another aspect of the invention in order to monitor the tissue temperature online and feedback to the physician information on the scanner (such as to the speed of scanning), the ice particle size or distribution of sizes, the kinetic energy of the ice particles or the distribution of the kinetic energy of ice particles, the direction of blowing, and more. Such a thermal image may be captured through a thermal camera or a CCD device which has some sensitivity in the infrared region. A combined visual image and thermal image may be produced on a monitor or in a special glasses worn by the physician.

Mixed particle populations having different characteristics or applied with different energy profile may provide a combined treatment of blasting and non-blasting effects on the skin simultaneously or interchangeably. The switch from one mode of operation to another mode of operation may be gradual so that the blasting effect is enhanced at the beginning when the SC is still in place and then slowly decreased until it stops when the SC has been removed and a prolonged cooling period is required.

For an application of fat removal, it is known in the prior art that cooling the fat layer to a temperature of −10° C. to +10° C. may cause the fat cells to crystallize or even create an apoptosis process. Therefore, according to this aspect of the invention, a system may be configured to manually or automatically expose a skin region, with or without a SC, to a blasting or non-blasting cooling profile using a beam of kinetically energized ice particles so that fat layer below such skin region may be maintained in a temperature range of −10° C. to +10° C. for a period of time of 1 sec to 1 hour.

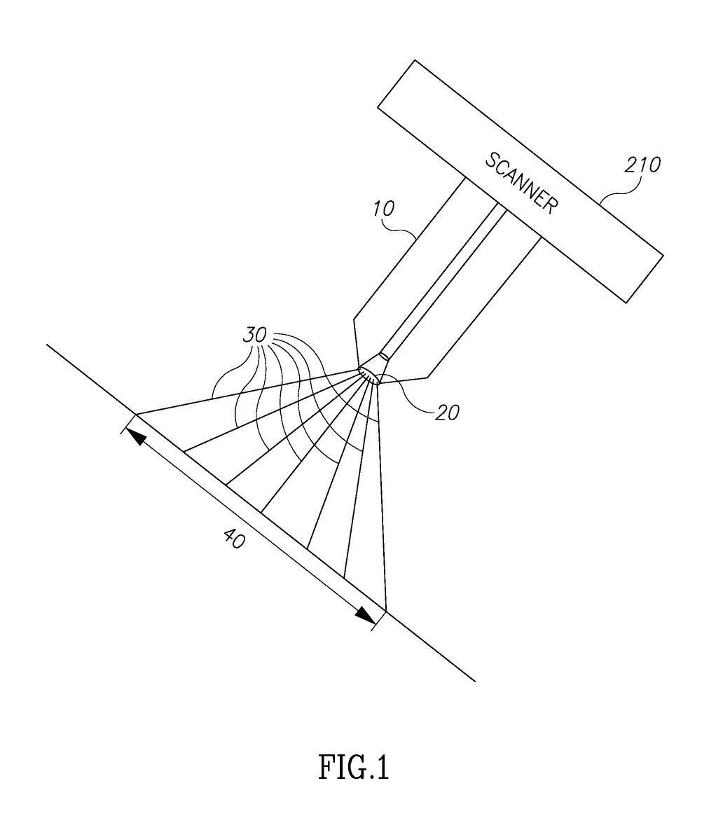

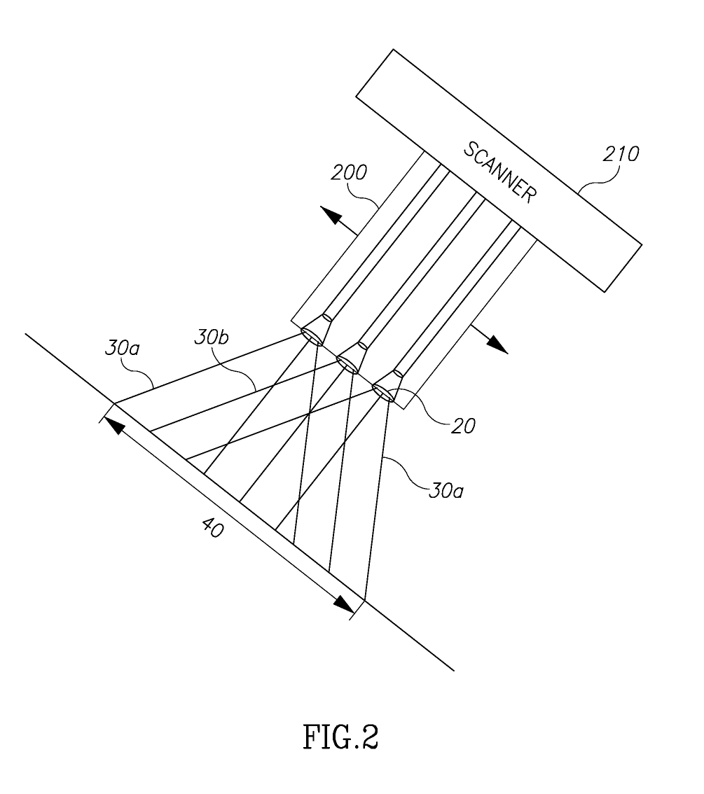

Different handpieces 10 and 200, scanner 210 and nozzle shapes 20 can be provided, such as a manual pen-like handpiece having a single nozzle or an array of nozzles configured to increase the flow of an ice crystal beam 30, 30a, 30b, its spot size 40, or to mix particles beams or populations, as shown in FIGS. 1 and 2.

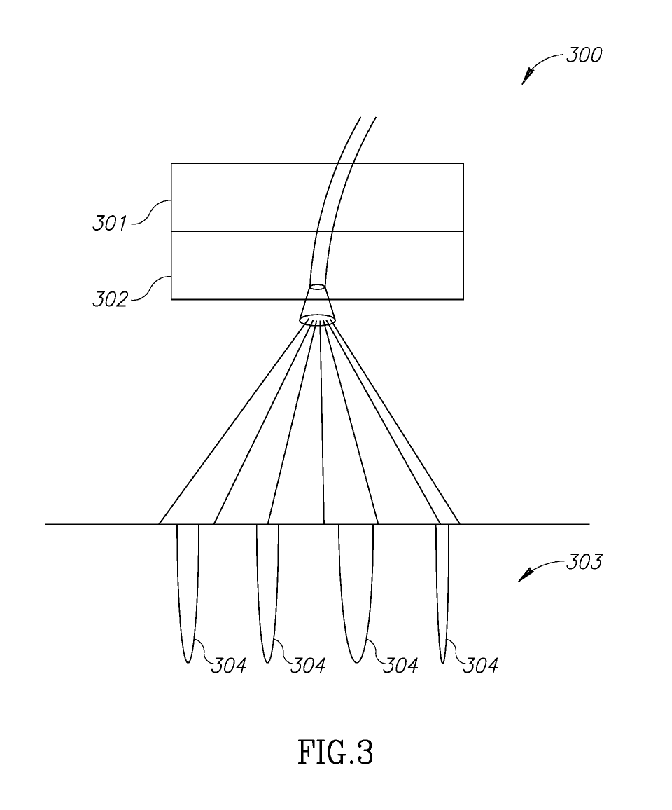

According to another aspect of the invention, as can be seen in FIG. 3, handpiece 300 may be combined with an ice blasting module 302 with, for example, a laser head 301 that may be configured to deliver for example a fractional skin tightening treatment to target tissue 303 causing tissue damaged zones 304. The ice crystals may come from shaving ice pellets or ice blocks or from compressed CO2 gas. Such a fractional treatment may be, for example, ablative or non-ablative treatment using a laser scanner or a beam splitter. Yet another advantage of using this technique is that the ice crystals will help cool the skin surface while the deeper layers of skin are treated. AS with the previously described embodiments as well as those to be described below, a programmed or programmable controller (not shown) may be used in conjunction with the various embodiments to control the operation of propelling the ice crystals, their velocity, their size, their distribution and frequency, orientation and timing as well as to control the application of laser (or other electromagnetic) energy before, during or after ice blasting.

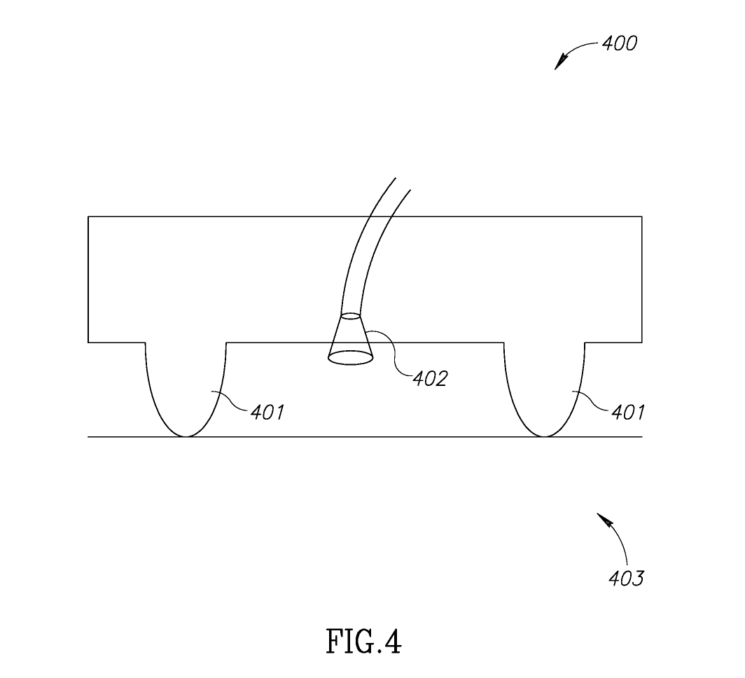

According to another aspect of the invention, as can be seen in FIG. 4, handpiece 400 may combine at least one RF or ultrasonic electrode 401 together with an ice handpiece having at least one ice nozzle 402 which is configured to treat tissue 403. According to the non-limiting example of FIG. 4, a bi-polar two or more electrodes configuration is disclosed where the ice treatment is configured to treat tissue layers before, during or after the RF or ultrasonic treatment of adjacent layers.

As known to those skilled in the art, there are a number of fractional lasers which may be used in accordance with this invention. Once such a laser may be a CO2 laser which is one of the common lasers in the industry. It is also known that dry ice sublimes and does so when it impinges the skin tissue rather than melting on the skin tissue. According to the combined laser ice treatment aspect of the invention mentioned above, there is a need to ensure that the laser energy is not absorbed in the ice particles or their vapors so that the optical energy can create the required tissue effect. Therefore, according to this aspect of the invention, in order to reduce the amount of laser energy absorbed in the CO2 gas which is vaporized from the dry ice particles the CO2 laser, it may be possible to use an isotopic CO2 laser using isotopic carbon (¹³C) in order to provide a wavelength of 11.2 micron instead of the standard CO2 laser wavelength of 10.6 micron. This will likely reduce the blooming effect of high absorption of the 10.6 micron laser in a CO2 gas environment.

### Dermabrasion Procedures

This is a known technique, and is used to remove and peel away skin layers until sufficient skin tissue wounding is achieved. The skin then starts a process of healing and grows a new layer of tight skin. Presently, such dermabrasion is performed using chemical peels as well as mechanical removal of the skin and often requires anesthetics to be delivered to reduce the pain that is experienced by the patient. However, dermabrasion can be provided using ice crystals impinging the skin. By changing the speed and size (or shape) and density of the ice crystals or pellets, deeper skin layers may be reached to cause the desired wounding. As with the stratum corneum removal discussed above, providing the treatment using ice crystals can provide skin cooling and pain reduction. As well, the ice crystals may not be pure CO2, but may be combined, when being formed, with medicines cosmetics, skin nutrition elements such as vitamins and minerals, nanoparticles such as Silver, Gold, Titanium dioxide, Zinc oxide, Quantum dots or Carbon based nanoparticles, or anesthetic substances to reduce pain and promote wound healing among other beneficial effects.

According to another aspect of the invention, as mentioned above, while the dry ice particles peel the upper layers of the skin, the remaining exposed skin portion is cooled and so are layers underneath. The cooled skin has reduced elasticity and it becomes more brittle. Therefore, the mechanical effect of the ice particles in terms of pealing is increased. According to another aspect, cold analgesic is provided to a treated tissue by the ice crystals to improve patient tolerance to ice or any other energy based treatment mentioned in this application.

Furthermore, the ice pellets that are “fired” towards the skin would touch the outer layer in a discontinuous manner and would bounce off the skin. This will allow the cooling of the skin layers but would prevent the damage caused by the prolonged physical contact of skin with dry ice. This method can be used for various applications, such as dry cooling of skin layers for pain management and for the selective cooling of the fat skin layers.

### Fractional Treatment Using Ice Crystals

As mentioned above, fractional treatment has been widely used with different energy based devices such as light/RF/US. The fractional treatment goal is to treat a fraction of the skin and generate deep channels of wounds in the skin. The untreated skin layers around the wounded channels will deliver the needed cells and components to start fast wound healing and collagen remodeling for skin tightening.

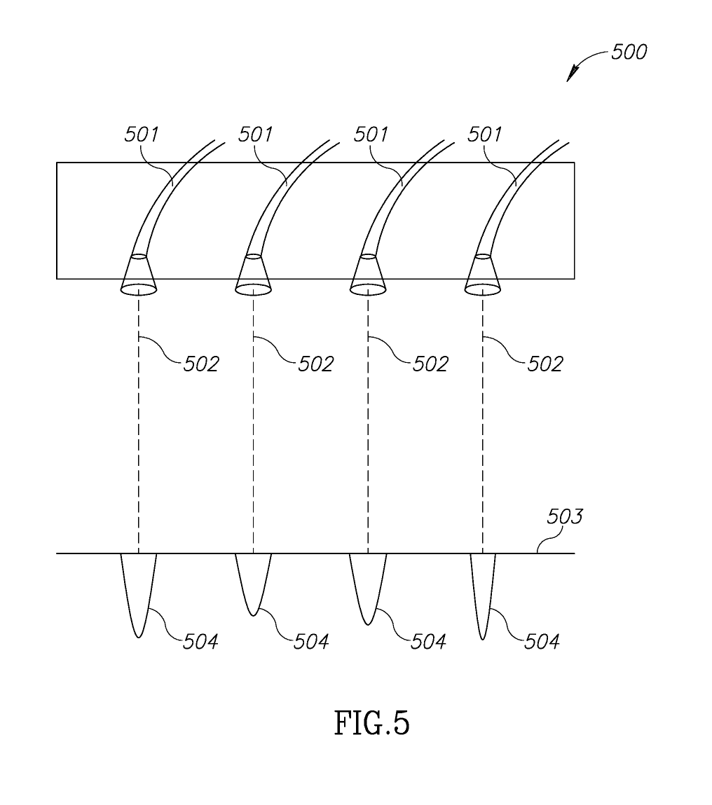

The same result can be achieved with ice crystals or pellets as seen in FIG. 5. In FIG. 5, it may be seen that handpiece 500 may have a series of spaced-apart nozzles 501 or other hollow tubes are provided that will “shoot” the ice crystals 502 into the skin surface 503. The penetration of the skin surface may be augmented by generating shaped pellets or crystals with one or more sharp ends, like a cone shape or the shape US football, that are propelled at a high speed and thus result in the delivery of high kinetic energy or by additives such as nanoparticles. The depth of the channels created 504 may be manipulated by changing the energy provided to the pellets for penetrating the skin surface, producing fractional effects, but with the added benefit of cooling of the skin surface and deeper tissue. Also, as mentioned above, the skin becomes brittle due to the reduction of the elasticity induced by the cold crystals; this may further increase the effect of the impact of the ice particles to create fractional channels in the tissue.

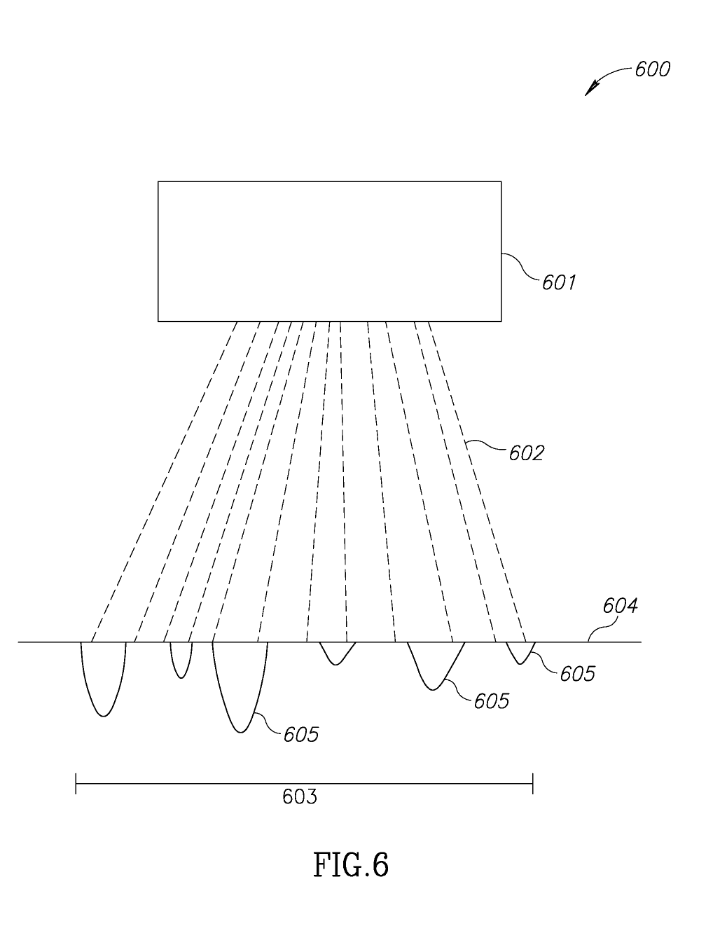

According to another embodiment of the present invention, as can be seen in FIG. 6, handpiece 600 may have ice blowing element 601 which is configured to generate dry ice particle stream, with or without additives such as nanoparticles, 602. Ice particle stream 602 may have a random dry ice spatial and or energy distribution creating stream spot of a size 603 on target tissue 604. As a result, random spatial distribution of channels may be created across the skin having a random depth and size. By controlling the particles population and their energies, some degree of the random tissue effect may be controlled too. One simple example may be two particles populations, one having an energy profile which is configured to create shallower holes while a second ice particle population has an energy profile which is configured to create a deeper hole in the tissue.

The process of treatment may start by creating ice crystals or pellets with a diameter of about 1 to about 50 μm. The diameter of the ice crystal or pellet will determine the diameter of the microscopic wounds or microchannels made to and into the skin surface. The ice crystals or pellets may be “fired” with high-speed at the skin surface using a handpiece with one nozzle or preferably a handpiece like that shown in FIG. 3 with a matrix of nozzles separated from each other by a fixed distance. The purpose of the fixed distance is to generate microscopic wounds or microchannels into the surface of the skin that are separated by some distance and by that targeting only a fraction of the skin of keeping the major part of the skin intact.

The firing of the ice crystals or pellets will be facilitated by one or more known devices that provide pressurization to give the ice crystals or pellets sufficient speed to penetrate the skin surface and into the skin tissue. The ice crystals or pellets will then enter the skin and start to sublime. The pellet's shape, mass and volume will be reduced and become smaller as it penetrates the skin layers, causing the microchannel to become narrower as the pellet goes deeper into the skin surface.

The depth of the microchannel may be determined by a number of factors, including the speed of the crystals or pellets and the number of pellets being fired, as more pellets will penetrate the channel and result in deeper wounding. The optimal range of a channel's depth is somewhere between 30 μm and increases to as much as 1000 μm.

Another benefit of using ice crystals of pellets is the evaporation of carbon dioxide inside the channels. It has been shown that exposing living cells to carbon dioxide will increase the blood vessel's diameter around the wounds, thereby delivering more oxygen to the treated region. This results in increasing metabolism, stem cells delivery and the necessary components for wound healing and skin revitalization. Cooling is also a side benefit of using ice crystals or pellets. Also, the delivery handle shown in FIGS. 5 and 6 may have included in it a fractional light/RF/US delivery device so that the ice crystal delivery may be combined with fractional light/FR/US delivery, simultaneously or alternately.

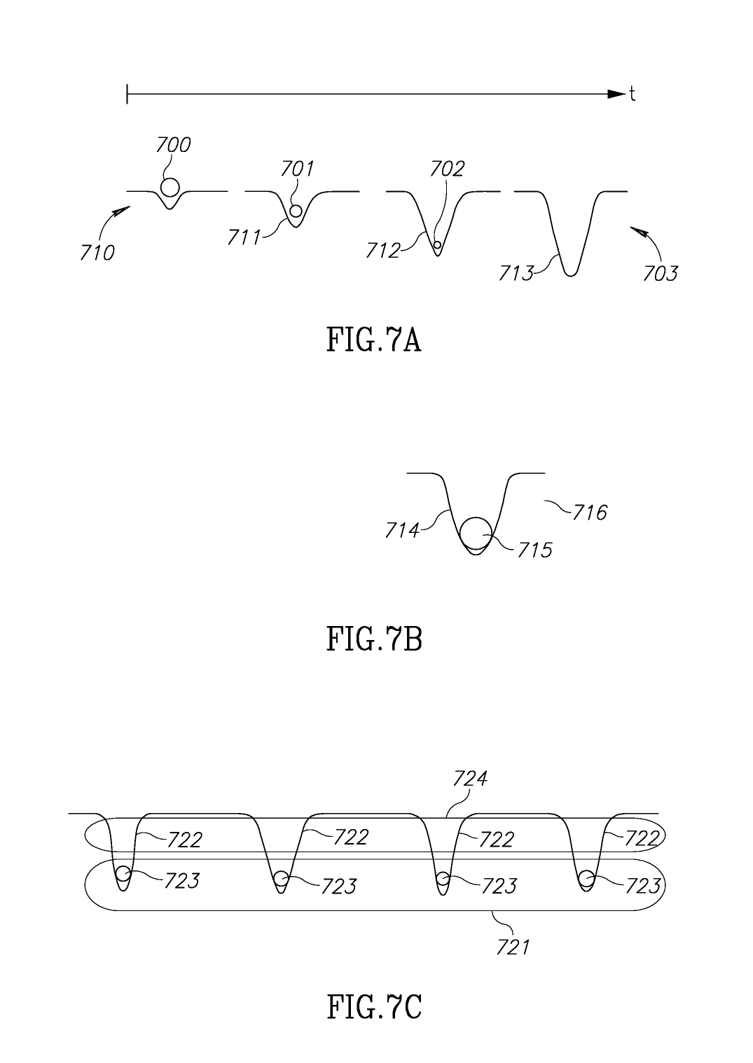

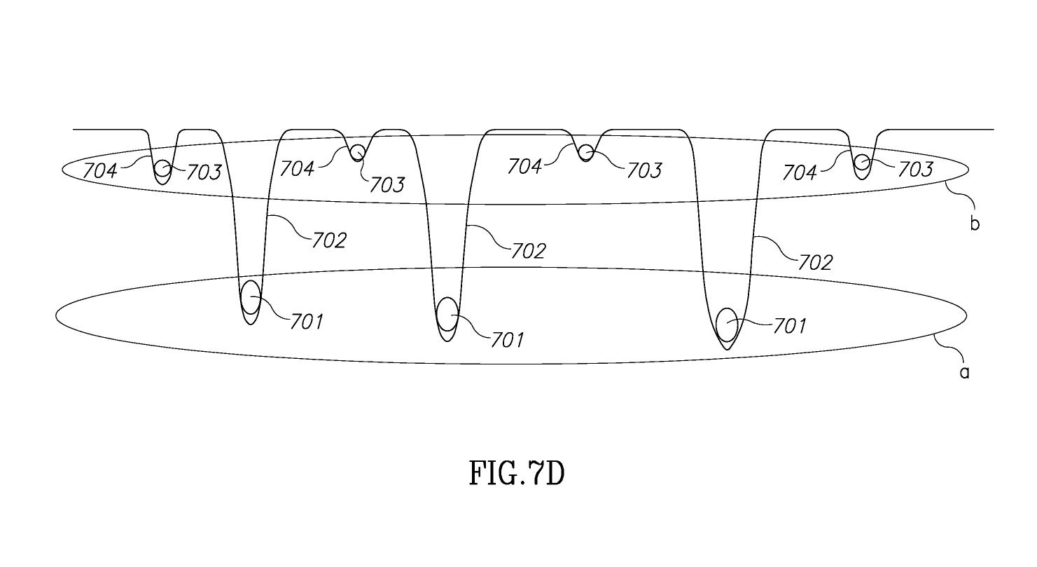

FIGS. 7a, 7b, 7c and 7d show different ice-tissue possible interactions along time axis t. In FIG. 7a tissue 703 is exposed to dry ice particle 700 creating tissue damage zone 710. As dry ice particle 700 loses kinetic energy and mass it becomes particle 701 in deeper tissue damaged zone 711. In the next step, ice particle 702 is smaller and having less kinetic energy in a tissue damaged zone 712. When the ice particle disappears, it leaves behind a tissue damage zone 713.

In FIG. 7b, tissue 716 is shown as having tissue damaged zone 714 as a result of an ice particle 715 having different energy characteristics. According to this example of the present invention, ice particle 715 has exhausted its kinetic energy before it has exhausted its mass. The end result is a tissue damage zone having a static dry ice mass at its bottom. The residual ice mass at the bottom of the fractional hole in the skin may act as a cooling source in the skin.

FIG. 7c shows the volumetric effect in volume 721 in skin 720 as a result of multiple skin damage zones 722 having residual ice mass 723 on their bottoms. As mentioned above, the residual element may be a nanoparticle and not necessarily an ice particle any more. According to this aspect, such a nanoparticle is a cooled nanoparticle and is remained cool when it reaches its target. Ice or residual nano particles 723 will reduce the temperature of the adjacent tissue in tissue volume 721 to a temperature which is lower that the temperature of tissue volume 724 above tissue volume 721. In other words, by seeding dry ice particles in a depth inside the tissue, a volume of tissue around this depth is cooled while the upper layers of tissue above this area may be maintained at a higher temperature. According to this aspect of the present invention, a thermal gradient across the skin may be achieved in which deeper layers of the tissue are colder than the upper layers of that tissue. As mentioned above, with topical cold application as known in the prior art it is impossible to achieve a similar temperature gradient without the use of needles. It should be mentioned that special needles like those available from Galil, which are configured to expose a target tissue to cold only at the distal end of the needle, are complicated to design and therefore cannot be produced as micro needles and in high density.

As can be seen in FIG. 7d, applying different particle populations can result in creating different layers parallel to the skin which have different temperature distribution. As shown in FIG. 7d, heavier dry ice particles 701 have a higher kinetic energy which allows deeper penetration into the tissue to produce tissue damaged zones 701 and also have a bigger residual mass. Particles 703 are characterized such that shallower tissue damaged zones 704 are created. The end result would be zone a′ having a lower temperature and zone b′ which is also a cooled volume but having a higher temperature than zone a′. The residual mass of the dry ice particles or nanoparticles additives dictate the time it takes the ice mass to sublime. Therefore, this dictates the temperature profile across the skin over time. For a longer cooling duration more mass is needed.

According to another aspect, one or more types of nanoparticles may be added to the dry ice particles. Different amounts of nanoparticles may be added to the dry ice particles based on the required density and amount of nanoparticles required to hit the target tissue. According to this aspect of the invention, the dry ice particles may be considered as carriers of the nanoparticles. As a carrier, the ice particles “buffer” allows a better way to control and propel the nanoparticles in the device on their way to the target tissue. Nanoparticles in the prior art are delivered to the skin as a paste or in the form of liquid droplets. Residual materials are therefore left on the target tissue. As mentioned above, dry ice particles tend to sublime when they hit a target leaving no or less residual material on the target. This allows the nanoparticles to interact directly and freely with an exposed skin (with or without SC). In this direct interaction of separated and free nanoparticles with the skin, nanofractional ablation of the target tissue may be achieved. Based on the amount of nanoparticles per dry ice particle carrier and based on the geometrical and energy profile of the dry ice carrier, it is possible to control the nanofractional effect of the tissue in terms of density of nanochannels, their size and depth. The nanoparticles, therefore, may cause mechanical nanofractional effects due to their mass, speed, density and penetration depth into the tissue. They may cause thermal effects due to the fact that despite their small size and small heat capacitance, they are delivered with a low temperature carrier so that they hit the target tissue while still having a temperature which is lower than the skin temperature, or lower than the room temperature or at about the temperature of the dry ice carrier particle. A third effect of the nanoparticles is based on the nature of the nanoparticle or nanoparticles used. For example, silver or copper nanoparticles may be used to achieve antibacterial effects in a target tissue or wound. Also according to this aspect, a two stage fractional system is described. In a first stage, a dry ice particle carrier is delivered to a target tissue. In a second stage, the dry ice particle hits a target tissue, sublimes and releases one or more nanoparticles to further hit, ablate, penetrate, peel or treat a target tissue. The temperature of the one or more nanoparticles is controlled by the temperature of the dry ice carrier and allows the release of the one or more nanoparticles adjacent a target tissue at a temperature similar to the temperature of the dry ice particle carrier. It should be mentioned that it is difficult and considered unsafe to release a stream of nanoparticles through the air from a reservoir directly to a target tissue without any carrier. Using dry ice particles as a carrier to nanoparticles may overcome these problems.

### Deliver of Drug or Other Medicinal Substances

As mentioned previously, the stratum corneum creates a strong barrier against material and molecule diffusion into the skin layers. Drug delivery into the skin layers can be facilitated with ice crystals or pellets by combining both processes. The drug can be either delivered right after the ice crystal or pellet delivery or simultaneously.

### Ice Crystal Treatment for Fat Reduction

It is known that the fat layer may be reduced by bringing the skin tissue to a range of 2-8° C. The fat droplets will crystalize and will then be removed by macrophages.

Ice crystals or pellets may be employed for the purpose of reaching the fat layers by loading them with enough high kinetic energy to create channels (as discussed above in the context of fractional treatment). The kinetic energy will be reduced to zero (because of the skin resistance) and the remaining pellet will remain in the channel to cool the area and result in the crystallization of the fat droplets.

This process can be generalized to not only treat fat droplets but also to facilitate a localized cooling of the treated area without cooling all the skin layer. The advantage of this localized cooling is reduction of pain resulted by cooling the upper skin layers to very low temperatures in order to reach the deeper skin layers.

### Use of Ice crystals or Pellets in Wound Debridement

The chronic wounds market is a wide market waiting for revolutionary processes for debridement of wounds for skin renewal. The employment of propelled ice crystals may be used to remove the affected area with no mechanical or frictional procedures that result in heating the skin. The cold temperature of the ice can be used as a means to manage the pain of peeling away the dead skin. The CO2 absorbed by the tissue may also contribute to the wound healing process, as mentioned above, by signaling the body to increase blood perfusion in the area. This is known as the Bohr effect.

### Other Uses of Propelled Ice Crystals or Pellets

These include: skin whitening, tattoo removal, selective hair removal, and even acne treatment and treatment of hyperhidrosis, as it has been found that sweat glands are very much affected by the temperature. Lower temperatures may reduce or even eliminate secretions from the glands. As mentioned above, a side benefit is cooling of the skin surface during any of the above discussed procedures, as well as the benefits of combining ice crystal or pellet treatments with light/RF/US treatments.

### Ice Crystals or Pellets in Brachytherapy-type Applications

While the above discussed the use of ice crystals or pellets which are propelled into the skin through the use of a sufficiently high energy source, another approach within the scope of the present invention is to adopt technology existing presently for the treatment of, for example, prostate cancer called brachytherapy. This cancer treatment technology involves the provision of one of more hollow needles through which very small pellets of radioactive particles are placed in the prostate. Adapting that technology to fat reduction, small ice crystals or pellets may be propelled deep into the skin tissue after the needles have been inserted into the patient's tissue Existing devices may be adapted and configured to seed ice particles into the fat tissue much as in brachytherapy systems radioactive seeds are placed in a cancers prostate tissue.

An additional advantage is that the technology already existing in the field of brachytherapy may be adopted to a fat reduction regime, including imaging systems to image a fat region and to control a planned treatment, a 3-D treatment planner to calculate temperature versus time profiles required to body sculpture specific area, determining in which places to seed the ice crystals or pellets and determining what should be the size of the ice crystals or pellet size and distribution. A device, which may be attached to a suitable handpiece, mounts one or more hollow needles to load a series of ice crystals or pellets into the hollow needles for delivery into the skin tissue. In addition, this same device may be utilized not only for the treatment of fat but also the treatment of prostate cancer, as a cryotherapy regime for the treatment of prostate cancer presently exists and is presently used in the treatment of certain types of prostate cancer.

As brachytherapy is practiced in two modes—HDT and LDT, ice brachycooling may also be practiced in similar ways. The equivalent to the HDT mode may be an ice particle loader coupled to an ice particle delivery tube having a rigid needle in its distal end. Ice may be loaded into the ice tube and the needle and inserted into a target tissue. The loader may be configured to move the ice particle along a certain region of the needle, the region inside the target tissue, so that heat is transferred through the needle wall from the target tissue. According to this method of the tissue treatment, no ice is seeded in the tissue. According to the equivalent of the LDT mode, the needles are configured to seed ice particles in the target region.

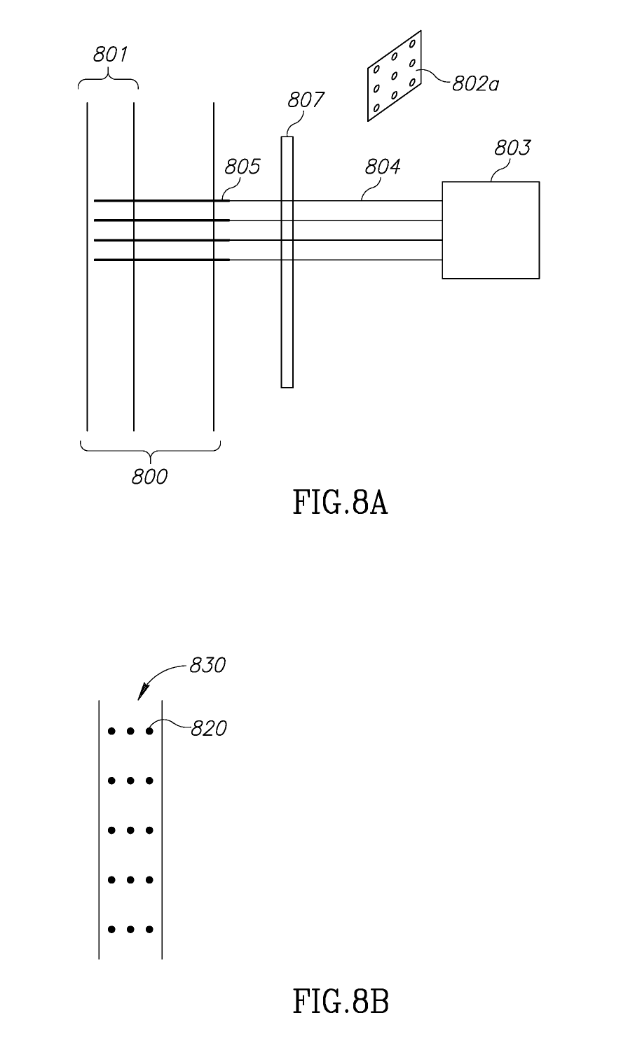

As can be seen in FIG. 8a, ice particle loader 803 is coupled to ice delivery tubes 804 having solid needles 805 on the distal end. A solid set of holes 802 (this is a side view) and is shown also as 802a) is placed in front of a target tissue to control the spatial distribution of solid needles 805 as they penetrate the target tissue, here which may be fat layer 801 in skin area 800. As mentioned above, a treatment plan is generated prior the treatment based on an ultrasound or other imaging system which provides an image of the location, contour and size of the tissue layer to be treated. Based on the image, a thermal analysis of the target tissue volume is performed and a treatment plan is generated defining the distribution and size of the ice particles to be seeded in order to produce the required thermal affect in the tissue. The ice particle loader is configured to retract each needle backwards a predefined distance in order to deposit the next ice “seed”. By doing so, as shown in FIG. 8b, an array of ice particles 820 can be seeded in a target tissue 830. The size and density of the ice particles dictates the cooling profile of the target tissue. When dry ice pellets are positioned in the subcutaneous skin layer it will lead to the crystallization of the lipid droplets and the death of fat cells. By bypassing the first skin layers no damage will be caused to the dermis and epidermis and thus the skin will be left intact. However, the fat tissue will be reduced from the subcutaneous region by controlled immune system and macrophages activity.

### Fat Tumors (Lipoma) Treatment

A fat tumor, known as a lipoma, is a benign tumor composed of adipose tissue. These lipoma tumors are often small (˜1 cm) but can sometimes reach a size of 6 cm., the tumor is often visible, movable, but painless. Due to the fact that it is a benign tumor, treatment is not necessary unless the tumor becomes painful and restricts movement. The tumors are usually surgically removed for cosmetic reasons. However, majority of surgeries performed today result in a visible scar.

Fat cell crystallization occurs prior to other cell types. This property will allow selective treatment of the lipoma tumors using ice pellets or crystals. The ice pellets will be fired using any of the handles mentioned above to the location of the tumor. The treatment can occur in two different methods. In the first method the pellets will be fired to the location of the tumor with low kinetic energy that will not allow skin layers' penetration. The pellets will cool the layers of the skin where the layer of the fat will selectively crystalize and be eliminated by macrophages. In the second method, the pellets will be fired in high kinetic energy to the location of the tumor and will penetrate the skin layers to reach the inner layers. The penetration of the pellets will generate channels in the skin layers. The ice pellets will remain in the end of the channel and will cool the surrounding tissue including the fat cells while the fat skin layers remain intact.

### Keloid-Scar Tissue Reduction

After the skin is injured, it usually forms a flat scar to protect the wound site. A keloid is defined as a scar tissue that grew excessively to sometimes reach a size greater than that of the wound. In many cases the Keloid may become itchy and painful. Treatment can be performed using cryosurgery or cryotherapy in which the keloid scar is frozen and this results in scar shrinkage.

Ice pellets or crystals can target keloid scars locally either by skin penetration or the firing of pellets onto the keloid scar. A handle for firing pellets can be located directly at the keloid scar. The fired pellets freeze the keloid and result in the death of skin cells around it and shrinkage of the scar. The ice pellets can either penetrate the skin or freeze the outer skin layer.

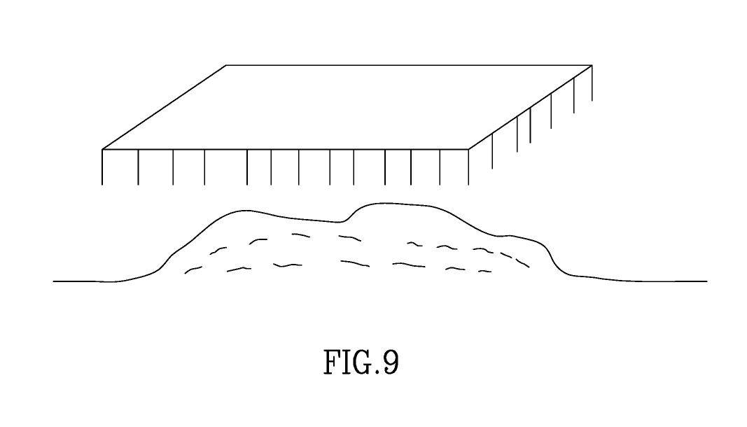

In addition, the firing of the ice pellets can be made to occur internally inside the keloid scar, as illustrated in FIG. 9, by using a sheet of needles that penetrate the keloid scar. The ice pellets are fired through the hollowed needles to the inner area of the keloid resulting in a homogenous freezing of the keloid scar.

### Hyperhidrosis

Excessive sweating is a common problem among human beings and can sometimes be related to thyroid problems, diabetes or infections. Treatments for excessive sweating include surgical operation for cutting or destroying nerves associated with active sweat glands located under the armpit. The surgery requires collapsing the lung in order to insert a catheter through the chest for nerve destroying and thus many complications arise related to this surgery.

Using ice pellets, the sweat glands or the nerves related to it can be targeted and destroyed. At least one needle is inserted at the armpit and reaches the sweat glands. The needles fires pellets to the location of the glands and selectively freeze sweat glands leading to death of the sweat glands.

1. A device which combines skin tissue cooling and microdermabrasion comprising:
a source of ice crystals;
a mechanism for propelling the ice crystals at a plurality of velocities;
a controller, the controller being programmable to increase or decrease the velocity of the propelled ice crystals;
wherein the controller controls the velocity of the ice crystals to range from low speed to impinge upon and cool the skin tissue to a higher velocity to cause microdermabrasion of the skin tissue.

2. The device for treatment of skin tissue of claim 1 comprising:
the mechanism for further one or more of: (a) sizing and shaping the ice crystals, and (b) fractionating the ice crystals delivery;
wherein the ice crystals are propelled, under the control of the controller, to impinge on the skin tissue surface in a spaced-apart, fractionated pattern.

3. The device of claim 2, wherein the controller controls the mechanism for propelling the velocity of the ice crystals impinging on the skin tissue with sufficient velocity to penetrate the skin tissue surface.

4. A device for the treatment of skin tissue comprising:
a source or ice crystals;
a mechanism for propelling the ice crystals;
a controller;
a source of electromagnetic energy in the vicinity of the mechanism for propelling the ice crystals;
wherein the controller controls: (a) the mechanism for propelling the ice crystals and (b) the source of electromagnetic energy;
wherein the controller causes the mechanism to propel ice crystals at the skin tissue one of before, during or after the source of electromagnetic energy is activated by the controller to impinge electromagnetic energy towards the skin tissue.

5. The device of claim 4, wherein the source of electromagnetic energy is one or more of: laser energy, RF energy and ultrasonic energy.

6. The device of claim 5, wherein the source of electromagnetic energy is fractionated energy.

7. A device for the treatment of tissue comprising:
one or more hollow needles;
a source of ice crystals, the ice crystals being sized to fit within the one or more hollow needles;
a controller;
a mechanism for propelling the ice crystals through the one or more hollow tubes;
wherein the controller causes the mechanism to propel the ice crystals through the one or more hollow needles, out the distal end of the one or more needles and into the tissue.
